(19) United States
(12) Patent Application Publication
(10) Pub. No.: US 2026/0002810 A1
(43) Pub. Date: Jan. 1, 2026

(54) CABLE BASED DETECTION OF BULK AGGREGATE

(71) Applicant: OPI Systems, Inc., Calgary (CA)

(72) Inventors: Khaled Saad, Calgary (CA); Cody Dalsin, Calgary (CA); Timothy Penrod, Calgary (CA); Adam Weiss, Calgary (CA)

(21) Appl. No.: 18/827,499

(22) Filed: Sep. 6, 2024

Related U.S. Application Data
(60) Provisional application No. 63/666,655, filed on Jul. 1, 2024.

(51) Int. Cl. G01F 23/292

(52) U.S. Cl. CPC G01F 23/2921

(57) ABSTRACT

Bulk aggregate management is provided. A method includes generating a spatial map corelating each multiple first sensors of one or more sensor cables to a location within a storage container for bulk aggregate. The method includes receiving, from the first sensors, a proximity detection signal. The method includes determining, from the proximity detection signal, a position of the bulk aggregate. The method includes generating control signals for conditioning the bulk aggregate.

### CROSS-REFERENCE TO RELATED APPLICATIONS

This application claims priority to U.S. Provisional Application Ser. No. 63/666,655, filed Jul. 1, 2024, which is incorporated by reference in its entirety.

### TECHNICAL FIELD

This disclosure generally relates to cable-based detection of bulk aggregate materials. For example, embodiments of the present disclosure may be employed in agricultural storage structures to monitor a condition of grain in a storage bin.

### BACKGROUND

Bulk aggregate materials, such as grains, seed, animal feed, or other agricultural products, may be stored in structures which may be referred to, generally, as bins or storage bins (to include grain elevators and silos). A condition of such aggregate materials can include a moisture content, temperature, volumetric quantity, insect activity, or so forth. Such a condition can be detected according to various sensors. For example, temperature cables have existed for more than eighty years. Temperature cables can be enmeshed into grain to detect hotspots. In some instances, cables enmeshed into the grain may include means to detect moisture. Controllers coupled with such sensors may be able to infer attributes of the grain including a fill level or moisture content. However, such inferences may rely upon changes to ambient conditions, as may unfold over many hours or even days. For this reason, some sensor suites include range-finding sensors such as plumb bobs, radar, LiDAR, or optical sensors to detect an upper surface of the grain. However, such sensors may be limited according to a line of sight. Such a limitation can result in delays or improper reporting of volume (and other conditions such as temperature, according to inferences relying on volumetric data). Further, non-detection of substantial voids in grain may inhibit other operations, owing to concerns of grain flows (sometimes referred to as grain avalanches).

### SUMMARY

The embodiments described herein attempt to overcome the deficiencies of conventional solutions by efficiently and quickly obtaining data. In one embodiment, a system for detecting a condition of a bulk aggregate may include a first sensor cable configured for disposition within the bulk aggregate. The first sensor cable includes multiple sensor sets spaced along an axial length, each sensor set including a proximity sensor to detect a presence of the bulk aggregate.

In some embodiments, the first sensor cable includes a uniform profile along the axial length. In some embodiments, the proximity sensors include an infrared sensor/emitter pair configured to detect the presence of the bulk aggregate. In some embodiments, the system includes further sensor cables, each including multiple further instances of the sensor sets along further axial lengths thereof. In some embodiments, the system includes a controller. The controller can communicatively couple with each of the sensor sets and further sensor sets. The controller can receive, from each of the sensor sets and further sensor sets, an indication of proximity of the bulk aggregate. The controller can determine, based on the indication of proximity, a position of the bulk aggregate. The controller can generate, based on the position, a first control signal configured to present an indication of the position. In some embodiments, each of the sensor sets include a humidity sensor, and the controller is configured to generate a second control signal configured to actuate a ventilation system to modulate a moisture content of the bulk aggregate based on a detected humidity.

In some embodiments, the sensors of each of the sensor sets are co-located at a sensor node. The sensor nodes may be spaced, at regular interval, along the first sensor cable. In some embodiments, each of the sensor sets include a temperature sensor.

In some embodiments, each of the sensor sets include a temperature sensor, and the controller is configured to generate a second control signal to condition the bulk aggregate based on temperature data received from the temperature sensor.

In another embodiment, a sensor cable may include multiple proximity sensors along an axial length. The proximity sensors are configured to detect a presence of a bulk aggregate abutting the sensor cable and provide, to a controller, an indication of a unique identity of a sensor and a proximity detection signal.

In some embodiments, each of the proximity sensors are organized into a sensor set, one or more of the sensor sets including a temperature sensor and a humidity sensor. In some embodiments, the proximity sensors are implemented as an emitter/receiver pair, and the proximity detection signal does not depend on a time of flight of a signal emitted by the emitter and received by the receiver.

An embodiment relates to a method for managing a bulk aggregate. The method may be executed by one or more processors of a controller. The method includes generating a spatial map corelating each multiple first sensors of one or more sensor cables to a location within a storage container for the bulk aggregate. The method includes receiving, from the first sensors, a proximity detection signal. The method includes determining, from the proximity detection signal, a position of the bulk aggregate. The method includes generating control signals. The control signals can be configured to display a state of fill of the bulk aggregate. The control signals can be configured to condition the bulk aggregate.

In some embodiments, the position of the bulk aggregate includes an interface between the bulk aggregate and a headspace of the storage container and a void within the bulk aggregate. In some embodiments, the bulk aggregate includes grain. In some embodiments, the one or more sensor cables are disposed in multiple concentric rings along a plane perpendicular to an axial length of the one or more sensor cables. The first sensors may be disposed, at regular interval, along the axial length of the one or more sensor cables. In some embodiments, the proximity detection signal is a return signal from an infrared emitter received at an infrared receiver of an emitter-receiver pair.

In some embodiments, the first sensors are disposed along an axial length of the one or more sensor cables in the storage container. The spatial map can include a unique identifier for each of the first sensors. In some embodiments, the control signals are configured to cause a display of a state of fill of the storage container. In some embodiments, the one or more sensor cables include multiple second sensors configured to indicate a humidity level. The method can include receiving, from the sensors, the humidity level and determining, based on the humidity level and the position of the bulk aggregate, a moisture content of the bulk aggregate. The control signals can actuate a ventilation system responsive to the moisture content. In some embodiments, the sensor cables are mechanically coupled with an upper surface of the storage container and a lower surface of the storage container. In some embodiments, the sensor cables include, along an axial length including the sensors, a uniform profile.

### BRIEF DESCRIPTION OF THE DRAWINGS

Various objects, aspects, features, and advantages of the disclosure will become more apparent and better understood by referring to the detailed description taken in conjunction with the accompanying drawings, in which reference characters identify corresponding elements throughout. In the drawings, like reference numbers generally indicate identical, functionally similar, and/or structurally similar elements.

FIG. 1 is an example of a sensor cable, according to some embodiments.

FIG. 2 is a block diagram for a data processing system, according to some embodiments.

FIG. 3 is a top view of a storage bin configured to receive a bulk aggregate, according to some embodiments.

FIG. 4 is a side view of a storage bin including various sensor cables, according to some embodiments.

FIG. 5 is a detail view of a front profile of a sensor node of a sensor cable, according to some embodiments.

FIG. 6 is a detail view of a side profile of a sensor node of a sensor cable, according to some embodiments.

FIG. 7 depicts a method for bulk aggregate management, according to some embodiments.

FIG. 8 is a block diagram illustrating an architecture for a computer system that can be employed to implement elements of the systems and methods described and illustrated herein.

The details of various embodiments of the methods and systems are set forth in the accompanying drawings and the description below.

### DETAILED DESCRIPTION

Systems and methods of the present disclosure employ proximity sensors to detect a presence of a bulk aggregate proximal to a sensor cable. For example, the bulk aggregate can include agricultural products such as grains or seeds. Indeed, some illustrative examples of the present disclosure are provided with reference to grain to illustrate some example implementations, as may aid the reader to understand certain of the benefits of the present disclosure; such illustrative examples should not be construed so as to limit the present disclosure.

The proximity sensors can include an emitter-receiver pair which emits a signal and, when in proximity to a bulk aggregate such as grain, receives a return signal as reflected by the grain and returned to the receiver. Various sensor nodes including proximity sensors may be disposed along the length of a sensor cable so that, depending upon operation of the various proximity sensors, one or more of the sensors may be determined to be “in grain” while others of the proximity sensors may be determined to be “out of grain.” Out of grain sensors can correspond to sensors disposed in a headspace at a top of a storage bin, or within voids formed in the grain. A controller coupled with the various sensors can, according to a predefined mapping with the location of the proximity sensors, map the position of the grain (e.g., to distinguish between in grain and out of grain sensors). Further, by receiving proximity data from multiple sensor cables, the controller can determine a profile of an interface layer between the headspace and the grain. For example, the controller can determine a presence or slope of a generally conical profile (as may formed from filling a bin), or an inverted cone (as may formed from emptying a bin) may be detected.

In some embodiments, the sensor cables can include temperature or humidity sensors. For example, the temperature or humidity sensors may be disposed separately from the proximity sensors along the cable, or may be co-located therewith at sensor nodes. Temperature and humidity data received from the sensors can indicate a condition of the grain (or other bulk aggregate). In some embodiments, the controller can communicate with various of the sensors to determine a condition, and communicate said condition to a user interface or conditioning equipment such as fans. For example, a condition can include a position of the grain (e.g., a height or profile of the interface layer, or a presence of any voids withing the grain). A condition can include an indication of temperature or moisture content of the grain. The controller can determine whether a measured temperature corresponds to a grain temperature, headspace temperature, or void temperature based on a detection of a proximity sensor. Likewise, the controller can determine a moisture content of grain based on a sensed relative humidity in combination with the presence of the grain. That is, the controller can determine either of the temperature or humidity of grain based on proximity data.

Determinations realized according to the systems and methods of the present disclosure can aid in the generation of improved accuracy, granularity, or time to first data, relative to other approaches which may attempt to algorithmically de-conflate in grain from out of grain sensors (e.g., relying on daily temperature fluctuations or humidity discontinuities as may arise between a headspace and grain). For example, in some embodiments, the controller is configured to automatically adjust a ventilation system based on the condition data (e.g., to actuate a fan or vent), wherein the actuation may aid in conditioning the grain. In some embodiments, the controller is configured to transmit a condition of the grain. For example, the controller can couple with a display device (e.g., a red/green light to indicate bin capacity, or an application for a mobile device).

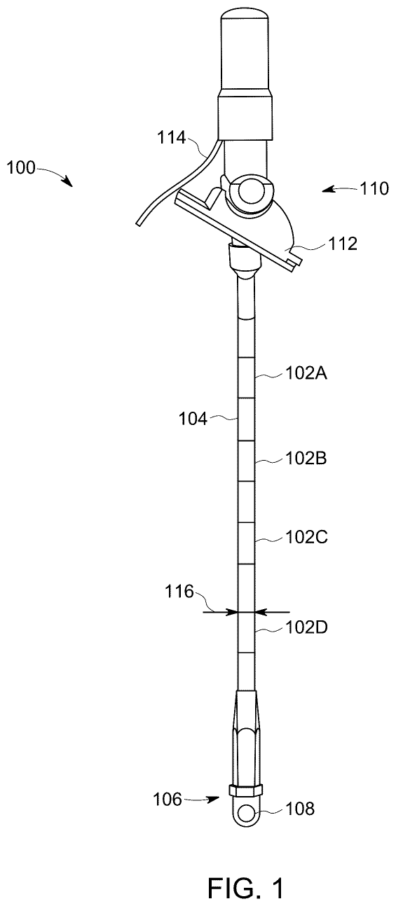

FIG. 1 is an example of a sensor cable 100, according to some embodiments. Like other figures provided herein, the sensor cable 100 is not necessarily depicted to scale. Indeed, certain features may be emphasized or de-emphasized to aid with the clarity of the figures. Many sensor cables 100 may extend for tens of meters, such as twenty meters, thirty meters, or fifty meters, although such illustrative examples should not be construed as limiting. The sensor cable 100 includes various instances of a sensor suite, as may be co-located at various sensor nodes and referred to as sensor sets 102A, 102B, 102C, 102D of one or more sensors. The depicted sensor sets 102A, 102B, 102C, 102D may be referred to collectively (or generally) as sensor sets 102. The sensor sets 102 may be mechanically coupled via a cable body 104. In some embodiments, the cable body 104 can include at least one power/signal line operatively coupling the various sensor sets 102 to one another, or to a controller.

Each sensor set 102 (depicted in FIG. 1 as a co-located sensor node) can include a proximity sensor to detect proximal bulk aggregate material such as grain or other agricultural products. In some embodiments, the sensor cable 100 can include further sensor sets 102 which omit the proximity sensor, but include other sensor types such as a temperatures sensor or a relative humidity sensor. For example, a sensor cable 100 can include alternating types of sensor sets 102, the alternating types including different sensor functions or transducer technologies. In some embodiments, any of the sensor sets 102 can include further sensors (e.g., the temperature sensor or relative humidity sensors). Every sensor set 102 includes at least one sensor; in some embodiments, the sensor sets 102 include multiple sensors (e.g., every sensor type of a cable).

The one or more sensors may be coupled with one another, or with a controller, via any of various architectures. For example, in some embodiments, each sensor set 102 includes of the sensor cable 100 includes communications circuity to communicate with a controller (e.g., via a common bussed connection). In some embodiments, a controller may couple with sensor sets 102 of multiple sensor cables 100, such as via a wired or wireless aggregation node.

A lower terminus 106 of the sensor cable 100 is adapted to couple with a lower surface of a storage bin. For example, the lower terminus 106 of the sensor cable 100 can include an eyelet 108 or other coupler to couple with the storage bin. In some embodiments, the lower terminus 106 is configured to receive a corresponding coupler (e.g., a tension line). In some embodiments, the sensor cable 100 can exhibit greater tensile strength than the corresponding coupler, such that the corresponding coupler can operate as a fused element.

An upper terminus 110 of the sensor cable 100 can be configured to couple with an upper surface of the storage bin. For example, the upper terminus 110 can include a mechanical coupler 112 configured to mechanically conform to an upper surface of the storage bin. The upper terminus 110 can further include electrical connections 114, such as a connection 114 with a solar panel or other power source, or a wired or wireless connections 114 for a controller (e.g., antennae or wired communication lines).

An axial length of the sensor cable 100 extends between the upper terminus 110 and the lower terminus 106. The axial length can correspond to a height of a storage bin, in some embodiments (although an adjustable length of the corresponding coupler, or deformation of the cable body 104 may account for minor deviations). Further, a flexibility of the cable body may conform to flows of a bulk aggregate (e.g., grain flows). A profile of the cable body 104 can be uniform along an axial length as may reduce tensile forces experienced according to such flows. For example, as is depicted, a one-dimensional width 116 of the sensor cable 100 is depicted as uniform along the axial extension of the two-dimensional depiction of the sensor cable 100 between the lower terminus 106 and the upper terminus 110.

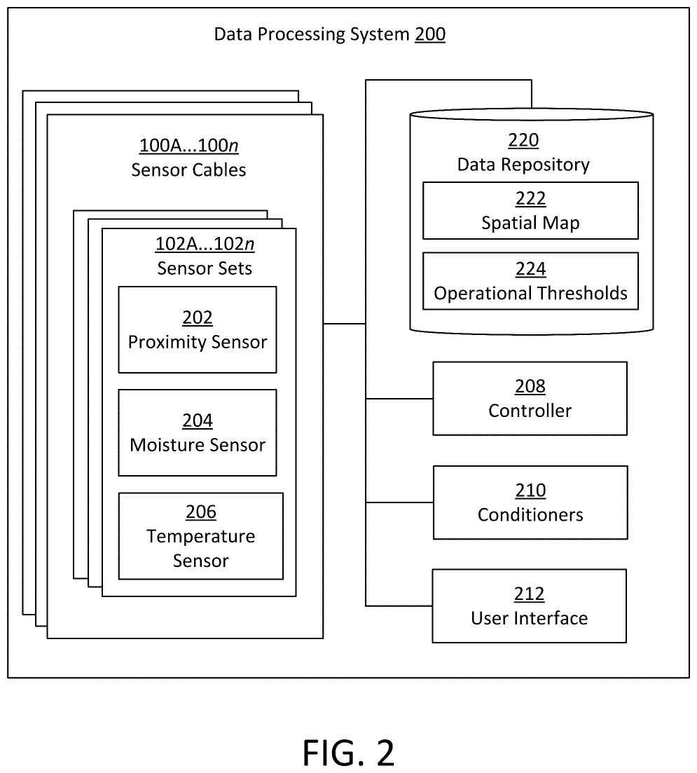

FIG. 2 is a block diagram for a data processing system 200, according to some embodiments. The data processing system 200 can include or interface with at least one sensor cable 100, each sensor cable 100 including any number of senser sets 102. For example, at least one of the sensor sets 102 can include proximity sensors 202 to detect a presence of a bulk aggregate proximal to the sensor cable 100. A same or further sensor set may further include sensors configured to detect a condition of the bulk aggregate (e.g., moisture sensors 204 or temperature sensors 206). The data processing system 200 can include or interface with at least one controller 208 communicatively coupled with the sensor sets 102, in some embodiments. The data processing system 200 can include or interface with at least one conditioner 210 (e.g., fan) to condition the bulk aggregate, in some embodiments. The data processing system 200 can include or interface with at least one user interface 212 to present information related to the condition of the bulk aggregate, in some embodiments.

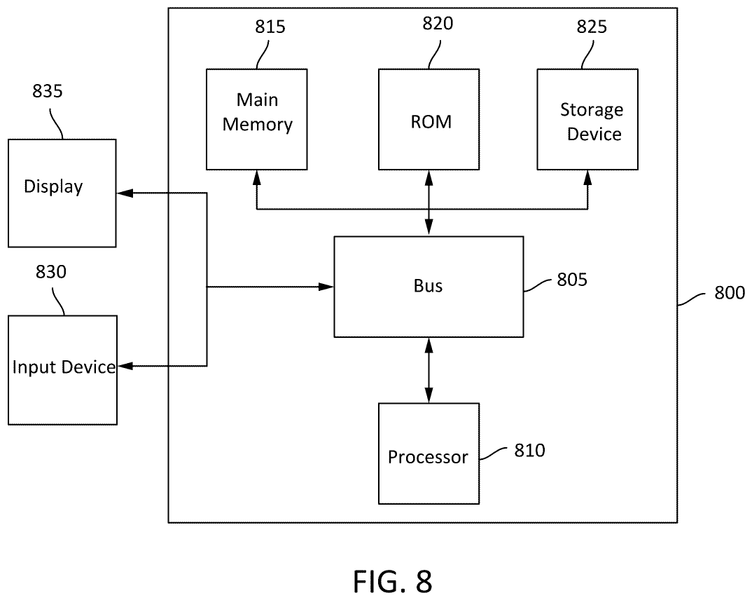

The sensor cables 100 (e.g., sensor sets 102 thereof), controllers 208, conditioners 210, or user interfaces 212 can each include at least one processing unit or other logic device such as programmable logic array engine, or module configured to communicate with the first data repository 220 or database. The sensor cables 100, controllers 208, or user interface 212 can be separate components, a single component, or part of the data processing system 200. The data processing system 200 can include hardware and software components to implement various functionality. For example, the hardware elements can include one or more processors, logic devices, circuits, or other components or structures of functionality of computing devices depicted in FIG. 8.

The data repository 220 can include one or more local or distributed databases, and can include a database management system. For example, the data repository 220 can be or include a local or cloud-based storage solution, which may be associated with a local instance of one or more sensor cables 100. The data repository 220 can store a spatial map 222 relating the sensors of the sensor cable 100 to a spatial location, or operational threshold 224 related to a storage bin, or bulk aggregate stored therein (as may be determined according to operation of the sensor cables 100).

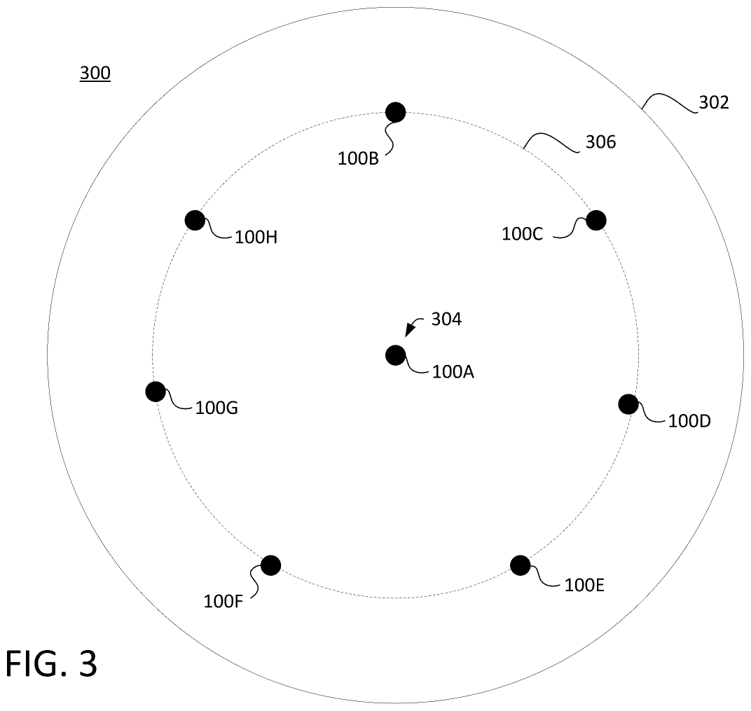

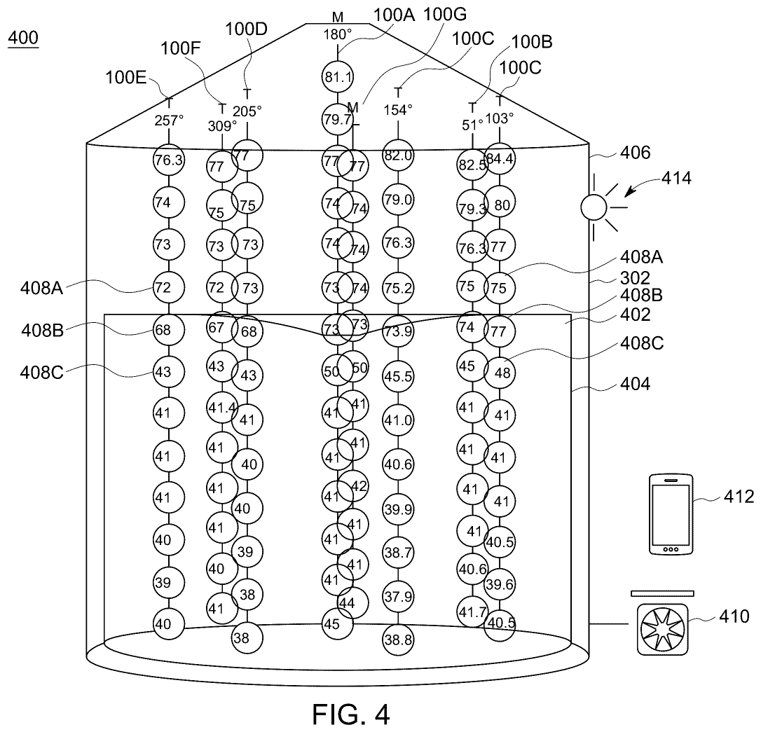

The spatial map 222 can correspond to a data structure mapping a location of various sensors of the sensor cable 100 to a physical location. For example, where sensors are disposed along an axial length of a sensor cable (e.g., at regular interval, such as every two or four feet), the spatial map 222 can include the sensor locations along the axial length of the sensor cable 100. The spatial map can correspond to a unique identifier for each sensor, or a set of sensors. In some embodiments including multiple sensor cables 100, the spatial map 222 can include a relative position between the various of the sensor cables 100. For example, the spatial map 222 can include a lateral offset perpendicular to the axial extension of the sensor cables (e.g., as depicted in FIG. 3). The spatial map 222 can include a vertical offset along the axial extension of the cables. For example, where multiple of the cables are affixed to a sloping roof of a storage bin, the vertical offset can corelate the relative heights of the upper terminals 110 of the sensor cables 100. A visual depiction of spatial map data is provided hereinafter, in FIG. 3 and FIG. 4.

The operational thresholds 224 can correspond to a condition of a bulk aggregate (e.g., agricultural products, such as grain) as may be stored in a storage bin. An operational threshold can correspond to a condition of the bulk aggregate, to include a position of an interface layer between the bulk aggregate and a headspace of the storage bin (e.g., a quantity of bulk aggregate), a temperature of the bulk aggregate, a moisture content of the bulk aggregate, a presence of any voids within the bulk aggregate, etc.

An operational threshold 224 can correspond to an automatic operation of other of the components of the data processing system 200. For example, one or more operational thresholds 224 can correspond to a moisture level of bulk aggregate (e.g., the controller 208 can execute an operation responsive to a detection of a moisture level exceeding a threshold). An operational threshold 224 can include a threshold for operation of a conditioner 210 such as a fan (e.g., the controller 208 can actuate the fan responsive to the moisture level). An operational threshold 224 can correspond a generation of a notification for presentation via a display. For example, one or more operational thresholds 224 can correspond to an indication of storage bin occupancy (e.g., full or empty indicator), overtemperature alerts, void indications, or so forth.

Each sensor set can refer to one or more sensors which are physically proximal along the length of a sensor cable 100 or logically related (e.g., coupled to a same processor of the controller 208). Although the depicted instance of the sensor set 102A depicts a set of a proximity sensor 202, moisture sensor 204, and temperature sensor 206, some sensor sets 102 can include additional or fewer sensors, such as sensors of further types, duplicated sensors (e.g., for redundancy purposes, implementing different transducer technologies, or having different fields of view). Although some examples of particular sensor types and descriptions of particular transducer implementations are provided below, the illustrative example should not be construed as limiting. The present disclosure contemplates the use of further sensor types or further transducer implementations (e.g., accelerometers to determine grain flows or coupler breakages, positional sensors to determine cable locations for the spatial maps, etc.).

A proximity sensor 202 can include an emitter/receiver pair such as an infrared emitter/receiver pair. In some embodiments, the emitter/receiver pair can operate according to another signal type such as a visual or ultrasonic signal. The emitter of the emitter/receiver pair may transmit a signal, as may be reflected by a bulk aggregate in proximity thereto. A receiver of the emitter/receiver pair can detect a reflection of the signal from bulk aggregate such as grain. In some implementations, the receiver does not determine a time-of-flight of the signal (e.g., to range the reflected signal). For example, such an implementation may reduce computational resources for the proximity sensor 202. For example, in some embodiments, infrared sensors can include a range of a few centimeters as may detect bulk aggregate directly abutting the sensor cable 100.

A moisture sensor 204 can detect a moisture content according to various sensor types. In some embodiments, the moisture sensor 204 includes a relative humidity (RH) sensor as may detect a modulation of an electrical capacitance or resistance, thermal conductivity, or other measured value between two nodes of the moisture sensor 204. The controller 208 can determine a moisture content of grain or other bulk aggregates based on the RH of air proximal to the bulk aggregate. For example, in a system at equilibrium, the controller 208 can determine that air proximal to the bulk aggregate is in moisture content equilibrium (EMC) with the air based on a number of time-series measurements within a threshold, and thus determine the moisture content according to the detected RH. In dynamic systems (e.g., when a fan is conditioning grain), the controller 208 can similarly determine the moisture content of the bulk aggregate based on an expected offset between the measured relative humidity and moisture content of the bulk aggregate. In some embodiments, the controller 208 can determine the expected offset based on a quantity or position of grain in the storage bin. In some embodiments, the controller 208 can determine the expected offset based on an operating condition or type of conditioning device (e.g., an airflow passed through the storage bin). In some embodiments, the moisture sensors can employ other transducers. For example, some sensor types may measure a moisture content of the bulk aggregate itself. For example, a moisture content of grain may be detected according to conductivity between the gran, near infrared spectroscopy, or so forth. In some instances, the moisture content is further determined based on temperature data received from a temperature sensor 206, such as by determining absolute humidity based on the sensed relative humidity and temperature data.

A temperature sensor 206 can detect a temperature of a bulk aggregate or air in a headspace. The temperature sensors 206 can include various types of transducers, such as resistance temperature sensors, thermistors (e.g., negative temperature coefficient thermistors), thermocouples, or so forth.

The controller 208 can couple with the proximity sensors 202, moisture sensors 204 or temperature sensors 206 to receive data therefrom. The controller can generate time-series data from one or more of the sensors. In some embodiments, the controller 208 can determine a condition of the grain based on the proximity sensor 202 in combination with further sensor data (e.g., moisture data received from the moisture sensors 204 or temperature data received from the temperature sensors 206). For example, the controller 208 can determine that a sensed temperature or moisture corresponds to an in-grain temperature or moisture based on proximity data received from the proximity sensor 202 proximal to the temperature sensor 206 (e.g., according to the spatial map 222).

The controller 208 can detect indicia of voids or other conditions (e.g., a presence of insects, molds or crusts in grain) according to a combination of the proximity data with humidity or temperature data. For example, such indicia may be identification according to a position of grain or voids. For example, an area showing no proximity return signal lower than the interface level of the grain may correspond to a void, while temperature or humidity anomalies detected below the interface level can correspond to mold, insect activity, or so forth. The controller can detect the anomalies according to a comparison between sensors data indicating a deviation exceeding operational thresholds 224 between the sensors or relative to an absolute value.

In some embodiments, the controller 208 can couple with sensors external to the storage bin, as may include local sensors or sensors for a remote data source (e.g., weather data). Such sensors can include temperature sensors 206, humidity sensors, windspeed sensors, or so forth. The controller 208 can determine a condition of the bulk aggregate based on sensor data of the external sensors (e.g., the weather data).

A conditioner 210 can include any device to condition a bulk aggregate. For example, a conditioner 210 can include a ventilation system of the storage bin. The ventilation system can include an active ventilation system such as fan (e.g., blower), or can operate based on convection via actuatable vents. The actuation of the ventilation system can modulate a temperature or moisture content of the bulk aggregate by exchanging air from the headspace or the bulk aggregate with air exterior to the storage bin. For example, to reduce a moisture content, the ventilation system can exhaust relatively moist air from the storage bin, and replace the exhausted air with relatively dry ambient air. Similarly, the controller 208 can modulate temperature or other conditions of the bulk aggregate by exchanging air. In some embodiments, the ventilation system includes an aeration system configured to pass air through the bulk aggregate. In some embodiments, the ventilation system includes a roof vents, side vents, etc., any of which may be actuated via control signals generated by the controller 208 responsive to comparisons of sensor data with the operational thresholds.

In some embodiments, a conditioner 210 can modulate a condition of grain or another bulk aggregate via a heater, fumigation system, auger, fire suppression system, or other component as the controller 208 may actuate responsive to a condition detected according to various received sensor data. In some embodiments, the controller 208 is configured to automatically actuate one or more conditioner 210 components responsive to a detected condition of the bulk aggregate. In some embodiments, the controller 208 is configured to present, via a user interface 212, an indication of the detected condition.

A user interface 212 can include any display (e.g., audio-visual display, audio display, or video display) to indicate a condition of the storage bin. In some embodiments, the user interface 212 includes one or more elements local to the storage bin. For example, the user interface 212 can include an indicator lamp (e.g., light emitting diode, LED) to indicate a quantity of bulk aggregate in a storage bin. The controller 208 can cause the indicator lamp to display an indication that a storage bin is full based on a detected position of bulk aggregate within the storage bin. For example, the controller can compare a fill level determined using proximity data and the spatial map 222 to an operational threshold 224 for fill level. Similarly, the controller 208 can cause an indicator lamp to indicate other conditions such as a presence of a void, moisture above or below an operational threshold 224, heat in excess of an operational threshold 224, or so forth. In some embodiments, the user interface 212 includes one or more elements remote from the storage bin. For example, the user interface 212 can include a display of a mobile device (e.g., a mobile application or web browser). The display can be configured to present notifications received from the controller 208 relating to the condition of the bulk aggregate, or present a dashboard including various information related to the condition of the bulk aggregate. For example, the display can depict a GUI depicting the information of FIGS. 3-4.

The controller 208 can include communications circuitry to interface between the various components of the data processing system 200, or between the components of the data processing system 200 and other devices. In some embodiments, the communications circuitry includes a network interface configured to communicate with one or more instances of a computing device (e.g., a mobile phone or a computer) in network communication with the data processing system 200. A network coupled with the network interface can include any number of other wired or wireless networks. For example, the components can be joined by an Ethernet, Wi-Fi, cellular, or other network interfaces. The network can include various boundaries such as boundaries between devices, subnets, firewalled networks, or the like.

In some embodiments, the network can include multiple instances of the data processing system 200 as may correspond to multiple storage bins. For example, the networked instances of the data processing system 200 can each monitor or condition at least one storage bin. In some embodiments, the various networked instances of the data processing system 200 can exchange sensor data. In some embodiments, the exchanged sensor data may be used by at least one controller 208 to display, via the user interface 212, a consolidated dashboard including information from multiple of the storage bins. The example, the user interface can present individual or total quantities of grain, average moisture content, grain bins most suited for a loading or unloading operation, a presence of mold or insets in various of the storage bins, an angle of repose, or so forth.

In some embodiments, the controller 208 can couple with further external data sources (e.g., weather forecasts or futures contracts process related to products stored in the storage bins). The controller 208 can present, via the user interface 212, data based on a combination of the external data and the condition of the bulk aggregate.

FIG. 3 is a top view 300 of a storage bin 302 configured to receive a bulk aggregate, according to some embodiments. For example, the storage bin 302 can include an entry door to load grain, and an outlet chute to discharge stored grain. According to loading and unloading of grain (or other bulk aggregates, according to various embodiments of the present disclosure), an interface level can form between the grain and a headspace above the grain. The interface may be generally level in a relatively flowable grain, or appear more conical according to an angle of repose of a less flowable grain. Further, some grains may form crusts of molds, leading to irregular non-conical patterns along the interface.

Further depicted are a first concentric ring 304 and second concentric ring 306 of one or more sensor cables 100. In some embodiments, a storage bin 302 can include additional or fewer concentric rings, or different arrangements of the sensor cables 100. The concentric rings are provided as an indication of the positions of the sensor cable 100, and should not be construed to necessarily correspond to a physical structure. The first concentric ring 304 includes a single, first sensor cable 100A. The second concentric ring 306 includes seven sensor cables 100 disposed as regularly spaced in the second concentric ring 306. For example, the seven concentric rings are disposed about 51.4° from one another around the second concentric ring 306. The controller 208 can map the position of the sensor cables 100 according to their angular position (e.g., 51°, 103°, 154°, etc.) or another indication of position. In some embodiments, the spatial map 222 can store a lateral location of a sensor cable 100 according to a selected ring (e.g., a diameter thereof) and a position along the ring.

Other concentric rings may include different numbers of sensor cables 100. A number of sensor cables may be selected to maintain a lateral distance between the sensor cables 100. For example, the distance between a second sensor cable 100B, third sensor cable 100C, fourth sensor cable 100D, fifth sensor cable 100E, sixth sensor cable 100F, seventh sensor cable 100G, or eighth sensor cable 100H of the second concentric ring 306 can be equal or similar to a distance between the first sensor cable 100A and any of the cable sensors of the second concentric ring 306. Similarly, each sensor cable 100 can include sensors spaced along an axial length thereof (e.g., into the page according to the present view).

FIG. 4 is a side view 400 of a storage bin 302 including various sensor cables, according to some embodiments. For example, the side view 400 can indicate a same or similar storage bin as is depicted in FIG. 3. An interface 402 defines the boundary between bulk aggregate 404 occupying a portion of the storage bin 302 and a headspace 406 of the storage bin 302. Each of the sensor cables 100 include various nodes 408 as may include at least one sensor type. In some embodiments, the depicted nodes 408 each correspond to a sensor set 102. According to a position of the interface 402, A first set of nodes 408A may be disposed in a headspace 406; a second set of nodes 408B may be disposed about the interface 402; and a third set of nodes 408C may be disposed in a bulk aggregate (e.g., in-grain).

A conditioner 210 coupled with the storage bin 302 includes a fan 410 configured to circulate air between the environment surrounding the storage bin and the bulk aggregate. The fan 410 (or other conditioner 210 components not depicted) may be automatically engaged by a controller 208 coupled therewith based on sensor data received from the sensor cables 100. Further depicted are user interface 212 instances, such as a mobile device 412 and indicator lamp 414 as may be coupled with the controller via its communications circuitry.

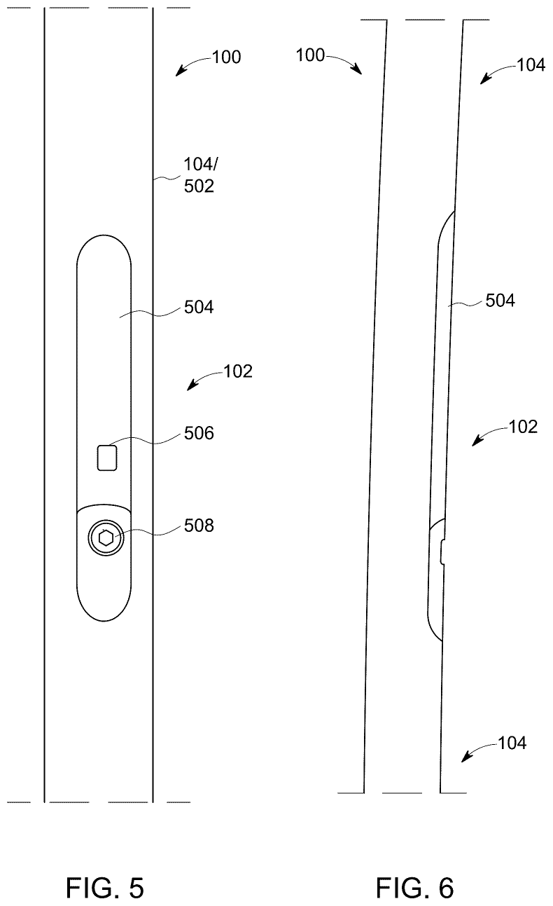

FIG. 5 is a detail view of a front profile of a sensor node 408 of a sensor cable 100, according to some embodiments. A cable body 104 can include a sheathing 502 as may be configured to interface with a bulk aggregate 404. For example, the sheathing 502 may be resistant to abrasion, adhesion, or chemical interactions with one or more bulk aggregate materials. The sheathing 502 includes an opening 504 corresponding to a sensor node 408 including one or more sensor types (e.g., at least a proximity sensor 202). In some embodiments, where a sheathing is sufficiently transparent to one or more sensors, the opening 504 may be omitted.

The opening 504 includes a window 506 to operatively couple a transducer of one or more sensors with an environment exterior to the sensor cable 100. For example, a window 506 for an infrared proximity sensor 202 can include an infrared transparent material such as sapphire or calcium fluoride. A different material may be selected for another transducer type such as an ultrasonic sensor (e.g., a material which is relatively transparent to ultrasonic pressure waves and otherwise resistive to contaminants, abrasion, or the bulk aggregate 404). A window 506 for a humidity sensor can include a material which is permeable to water vapor while being impermeable to the bulk aggregate, contaminants, liquid water, or so forth. For example, such a window can include polytetrafluoroethylene (PTFE), polypropylene (PP), or various polymer films.

In some embodiments, the sensor cable 100 includes an access panel as may be accessible via a fastener 508. Further, in some embodiments, the fastener 508 may couple sections of the sensor cable 100 to adjust a selected length or number of sensor sets 102. In some embodiments, the sensor cable 100 may rotate about the fastener to deform to bulk aggregate flows, or other portions of the sensor cable (e.g., a cable body 104) can be deformable.

FIG. 6 is a detail view of a side profile of a sensor node 408 of a sensor cable, according to some embodiments. The side profile may be of differing dimensions from the front profile (e.g., may be generally elliptical, such as D-shaped). The profile can receive a circuit board, sensor, controller 208, or other component of the sensor node 408 while reducing a cross sectional dimension of the cable (so as to reduce forces experienced by the sensor cable 100 incident to bulk aggregate flows). In other embodiments, the sensor cable 100 may be substantially circular. In any case, a profile of the cable may be uniform as may reduce forces experienced by the sensor cable 100 during bulk aggregate flows, relative to other approaches. For example, such bulk aggregate flows can include bulk aggregate mixing, loading, unloading, settling, or other operations.

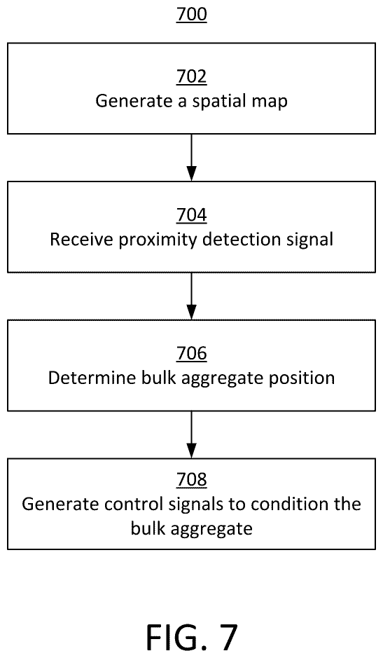

FIG. 7 depicts a method 700 for bulk aggregate management, according to some embodiments. The method 700 can be performed by one or more systems or components depicted herein (e.g., the data processing system 200 of FIG. 2, such as the controller 208 thereof, the controller including one or more processors). In some embodiments, the method 700 may be performed by a controller of an aggregation box configured to couple with various sensors, as may be included in one or more sensor cables, or otherwise arrayed to collect sensor data from a storage bin. The method 700 can include additional, fewer, or different operations according to various embodiments.

At operation 702, the data processing system 200 generates a spatial map 222 corelating sensors of at least one sensor cable 100 to locations disposed within a storage container for a bulk aggregate. In some embodiments, the bulk aggregate is grain, and the storage container is a grain bin. The one or more sensor cables may be disposed in multiple concentric rings along a plane perpendicular to an axial length of the sensor cables in the storage container. Sensors may be disposed, at regular interval, along the axial length of the sensor cables. In some embodiments, the cable sensors include various sensor types as may vary along the length of the sensor cable 100. For example, a cable sensor 100 can include a temperature sensor in every third sensor set, or omit a proximity sensor from some sensor sets. The sensor cables may be mechanically coupled with an upper surface of the storage container (at an upper terminus 110 of the sensor cable 100) and a lower surface of the storage container (at a lower terminus 106 of the sensor cable 100).

At operation 704, the data processing system 200 receives, from the sensors, a proximity detection signal. For example, the proximity detection signal may be received as a return signal from an emitter received at a receiver of an emitter-receiver pair (e.g., an infrared emitter-receiver pair). In some embodiments, the sensor cables include further sensors, such as sensors configured to indicate a humidity level. The controller 208 can receive, from such sensors, the humidity level. The controller 208 can determine, based on the humidity level and the position of the bulk aggregate, a moisture content of the bulk aggregate. The controller 208 can generate control signals configured to actuate a ventilation system responsive to the moisture content (e.g., to dry grain).

In some embodiments, the various sensor data is received from a sensor set including multiple sensor types (e.g., a same sensor set including a proximity sensor, humidity sensors, and temperature sensors). In some embodiments, the various sensor data is received from separate sensor sets. For example, proximity data may be received from a sensor set including a proximity sensor, while humidity data may be received from another sensor set including a humidity sensor.

At operation 706, the data processing system 200 determines, from the proximity detection signal, a position of the bulk aggregate. The determination of the position can include a determination of an interface between the bulk aggregate and a headspace 406 of the storage container (e.g., an average level, quantity of stored grain, angle of repose, or indication of crusting or mold, such as radial irregularities in the angle of repose). In some instances, the position of the bulk aggregate can indicate a void within the bulk aggregate, as may be detected by the controller 208.

At operation 708, the data processing system 200 generates control signals to condition the bulk aggregate. The control signals can actuate a conditioner 210 component (e.g., actuate a ventilation system responsive to the moisture content) or cause a display of a detected condition (e.g., a state of fill of the storage container, to include various aspects of the position as referred to at operation 706).

FIG. 8 is a block diagram illustrating an architecture for a computer system 800 that can be employed to implement elements of the systems and methods described and illustrated herein. The computer system or computing device 800 can include or be used to implement the controller 208 or its components, and components of the systems provided herein. The computing system 800 includes at least one bus 805 or other communication component for communicating information and at least one processor 810 or processing circuit coupled with the bus 805 for processing information. The computing system 800 can also include one or more processors 810 or processing circuits coupled with the bus for processing information. The computing system 800 also includes at least one main memory 815, such as a random-access memory (RAM) or other dynamic storage device, coupled with the bus 805 for storing information, and instructions to be executed by the processor 810. The main memory 815 can be used for storing information during execution of instructions by the processor 810. The computing system 800 can further include at least one read only memory (ROM) 820 or other static storage device coupled with the bus 805 for storing static information and instructions for the processor 810. A storage device 825, such as a solid-state device, magnetic disk or optical disk, can be coupled with the bus 805 to persistently store information and instructions (e.g., for the data repository 220).

The computing system 800 can be coupled via the bus 805 to a display 835, such as a liquid crystal display, or active-matrix display. An input device 830, such as a keyboard or mouse can be coupled with the bus 805 for communicating information and commands to the processor 810. The input device 830 can include a touch screen display 835.

The processes, systems and methods described herein can be implemented by the computing system 800 in response to the processor 810 executing an arrangement of instructions contained in main memory 815. Such instructions can be read into main memory 815 from another computer-readable medium, such as the storage device 825. Execution of the arrangement of instructions contained in main memory 815 causes the computing system 800 to perform the illustrative processes described herein. One or more processors in a multi-processing arrangement can also be employed to execute the instructions contained in main memory 815. Hard-wired circuitry can be used in place of or in combination with software instructions together with the systems and methods described herein. Systems and methods described herein are not limited to any specific combination of hardware circuitry and software.

Although an example computing system has been described in FIG. 8, the subject matter including the operations described in this specification can be implemented in other types of digital electronic circuitry, or in computer software, firmware, or hardware, including the structures disclosed in this specification and their structural equivalents, or in combinations of one or more of them.

The foregoing method descriptions and the process flow diagrams are provided merely as illustrative examples and are not intended to require or imply that the steps of the various embodiments must be performed in the order presented. The steps in the foregoing embodiments may be performed in any order. Words such as “then,” “next,” etc. are not intended to limit the order of the steps; these words are simply used to guide the reader through the description of the methods. Although process flow diagrams may describe the operations as a sequential process, many of the operations may be performed in parallel or concurrently. In addition, the order of the operations may be re-arranged. A process may correspond to a method, a function, a procedure, a subroutine, a subprogram, and the like. When a process corresponds to a function, the process termination may correspond to a return of the function to a calling function or a main function.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’”' can include only ‘A’, only ‘B’, as well as both ‘A’ and ‘B’. Such references used in conjunction with “comprising” or other open terminology can include additional items.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware may be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the generic principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

1. A system for detecting a condition of a bulk aggregate, the system comprising a first sensor cable configured for disposition within the bulk aggregate, the first sensor cable comprising:
a plurality of sensor sets spaced along an axial length of the first sensor cable, each sensor set comprising a proximity sensor to detect a presence of the bulk aggregate along the axial length of the first sensor cable; and
a controller configured to:
communicatively couple with each of the sensor sets;
receive, from each of the sensor sets, an indication of proximity of the bulk aggregate;
determine, based on the indication of proximity, a position of the bulk aggregate, the indication comprising an indication of a void formed under an upper surface of the bulk aggregate; and
generate, based on the position, a first control signal configured to present an indication of the position of the bulk aggregate, wherein the bulk aggregate comprises the void.

2. The system of claim 1, wherein the first sensor cable comprises, along the axial length, a uniform profile.

3. The system of claim 1, wherein the sensors comprise proximity sensors having an infrared sensor/emitter pair configured to detect the presence of the bulk aggregate.

4. (canceled).

5. The system of claim 1, wherein:
each of the sensor sets further comprise a humidity sensor; and
the controller is configured to generate a second control signal configured to actuate a ventilation system to modulate a moisture content of the bulk aggregate based on a humidity signal received from the humidity sensor.

6. The system of claim 1, wherein:
each of the sensor sets further comprise a temperature sensor; and
the controller is configured to generate a second control signal configured to condition the bulk aggregate based on temperature data received from the temperature sensor.

7. The system of claim 5, wherein the proximity sensor of each of the sensor sets are co-located with at least a temperature sensor at a sensor node.

8. The system of claim 7, wherein the sensor nodes are spaced, at regular interval, along the first sensor cable.

9. A sensor cable assembly comprising:
a plurality of proximity sensors disposed along an axial length of the sensor cable, the plurality of proximity sensors configured to:
detect a presence of a bulk aggregate abutting the sensor cable; and
provide, to a controller, an indication of a unique identity of a sensor and a proximity detection signal; and
the controller configured to:
communicatively couple with each of the proximity sensors;
receive, from each of the proximity sensors, an indication of proximity of the bulk aggregate;
determine, based on the indication of proximity, a position of the bulk aggregate, the indication comprising an indication of a void formed under an upper surface of the bulk aggregate; and
generate, based on the position, a first control signal configured to present an indication of the position of the bulk aggregate, wherein the bulk aggregate comprises the void.

10. The sensor cable of claim 9, wherein each of the plurality of proximity sensors are organized into a sensor set, one or more of the sensor sets comprising:
a temperature sensor; or
a humidity sensor.

11. The sensor cable of claim 10, wherein the proximity sensors are implemented as an emitter/receiver pair, and the proximity detection signal does not depend on a time of flight of a signal emitted by the emitter and received by the receiver.

12. A method for managing a bulk aggregate, the method comprising:
generating, by one or more processors, a spatial map corelating each of a plurality of first sensors of one or more sensor cables to a location disposed within a storage container for the bulk aggregate;
receiving, by the one or more processors, from the plurality of first sensors, a proximity detection signal;
determining, by the one or more processors from the proximity detection signal, a position of the bulk aggregate comprising an interface between the bulk aggregate and a headspace of the storage container and a void within the bulk aggregate; and
generating, by the one or more processors, control signals configured to cause a display of a state of fill of the storage container, the state of fill comprising an indication of the position of the bulk aggregate comprising the void.

13. (canceled)

14. The method of claim 12, wherein:
the bulk aggregate comprises grain;
the one or more sensor cables are disposed in two or more concentric rings along a plane perpendicular to an axial length of the one or more sensor cables; and
the plurality of first sensors are disposed, at regular interval, along the axial length of the one or more sensor cables.

15. The method of claim 12, wherein the proximity detection signal is a return signal from an infrared emitter received at an infrared receiver of an emitter-receiver pair.

16. The method of claim 12, wherein:
the plurality of first sensors are disposed along an axial length of the one or more sensor cables disposed in the storage container; and
the spatial map comprises a unique identifier for each of the first sensors.

17. (canceled)

18. The method of claim 16, wherein the one or more sensor cables comprise a plurality of second sensors configured to indicate a humidity level, and further comprising:
receiving, by the one or more processors from the second plurality of sensors, the humidity level; and
determining, by the one or more processors based on the humidity level and the position of the bulk aggregate, a moisture content of the bulk aggregate, wherein the control signals to actuate the conditioner are configured to actuate a ventilation system responsive to the moisture content.

19. The method of claim 18, wherein the one or more sensor cables are mechanically coupled with:
an upper surface of the storage container; and
a lower surface of the storage container.

20. The method of claim 18, wherein the one or more sensor cables comprise, along the axial length including the first sensors, a uniform profile.

21. The system of claim 1, wherein the presentation of the indication of the position of the bulk aggregate comprises a display of the indication of the position of the bulk aggregate.

22. The sensor cable assembly of claim 9, wherein the presentation of the indication of the position of the bulk aggregate comprises a display of the indication of the position of the bulk aggregate.
